(19) United States
(12) Patent Application Publication
(10) Pub. No.: US 2010/0211033 A1
(43) Pub. Date: Aug. 19, 2010

(54) TAP FOR A DRAINAGE BAG

(75) Inventor: John Blum, Toms River, NJ (US)

Correspondence Address: CONVATEC INC., 100 HEADQUARTERS PARK DRIVE, SKILLMAN, NJ, 08558, US

(73) Assignee: ConvaTec Technologies Inc.,, Skillman, NJ (US)

(21) Appl. No.: 12/668,727

(22) PCT Filed: Jul. 16, 2008
(86) PCT No.: PCT/US08/70168

§ 371 Date: Jan. 12, 2010

Related U.S. Application Data
(60) Provisional application No. 60/949,975, filed on Jul. 16, 2007.

(51) Int. Cl. B67D 3/04; A61F 5/44

(52) U.S. Cl. 604/335; 222/556

(57) ABSTRACT

A urine drainage bag, such as a urostomy bag, includes a drainage tap. The tap includes a tap member mounted to a housing so as to be pivotably movable between a closed, stowed position and an open, deployed position. In the closed position, the tap member is accommodated substantially within a chamber of the housing. The housing is disposed between front and rear walls of the bag, such that the tap is integrated into the seam of the bag. The tap further comprises a drip cap that is captively mounted on the tap member. A portion of the drip cap acts as a handle for pulling on the tap member. A portion of the drip cap also acts as a retainer or lock for blocking accidental closing of the tap member when attached to a night drainage apparatus.

### FIELD OF THE INVENTION

The present invention relates to a tap for a drainage bag, for example, a urine drainage bag. Such drainage bags include, but are not limited to, urostomy bags and urine leg bags.

### BACKGROUND TO THE INVENTION

Many designs of taps for drainage bags are known. However, some conventional designs suffer from one or more of the following problems: (i) the tap is rather bulky compared to the profile of the drainage bag; (ii) the tap is vulnerable to catching on the user's clothing, and opening accidentally, leading to leakage of urine onto the user's clothing; (iii) at night when the tap is opened and connected to a night drainage apparatus, the tap is vulnerable to closing accidentally by involuntary movement of the user while asleep, thus leading to backing-up of urine and risk of infection and overflow of urine; (iv) the tap does not permit complete drainage of liquid from the bag; and (v) the tap cannot be welded, in a pre-assembled condition, to a drainage bag using automatic apparatus. Taps suffering from this last problem cannot be properly tested prior to attachment to the bag because either the tap components have to be disassembled after testing in order to attach the tap, or the tap can only be tested once the tap components are assembled after attachment to the drainage bag. Moreover, assembly of the tap after attachment to the drainage bag is more complicated and expensive, since it is more difficult to manipulate the tap components.

U.S. Pat. No. 6,021,928 describes a tap that addresses problem (v). The tap includes an L-shaped tap member, one end of which is inserted into a base. The tap member is rotatable through 180 degrees between an open position in which the outlet points downwardly away from the base, and a closed position in which the outlet points upwardly and sits against the face of the base. The tap can be assembled into a finished tap unit, tested, and then attached to the drainage bag in the ready-assembled condition. The ability to test the tap before attachment to the drainage bag, and to attach the tap without having to disassemble the tap member, can improve manufacturing yield of the drainage bags, and reduce the number of manufactured bags that have to be discarded as a result of defective taps. It can also enable more efficient manufacture of the drainage bag.

The present invention has been devised to further enhance certain properties of drainage bag taps, bearing the above problems in mind.

### SUMMARY OF THE INVENTION

One aspect of the invention provides a tap for a drainage bag, the tap comprising a tap member mounted to a housing, the tap member being pivotably movable between an open, position and a closed position. The invention may further comprise one or more of the following features:

(a) The open position is a deployed position in which the tap member projects substantially from the housing. The closed position is a stowed position in which more of the tap member is received within the housing than in the open position.

By stowing the tap member within the housing when in its closed position, the tap member is protected by the housing. Therefore, the risk of the tap member catching on the wearer's clothing, and accidentally opening, is significantly reduced.

In one form, when in the closed position, the tap member is substantially entirely received within the housing. In an alternative form, some portion of the tap member may remain projecting from the housing in the closed position.

(b) The tap further comprises a retaining device for retaining the tap member in the open position and/or for obstructing movement of the tap member into the closed position. The retaining device may be activatable when the tap member is open, and deactivatable when it is desired to close the tap member. Additionally, or alternatively, the retaining device may be configured to obstruct closing of the tap member when a drainage adapter is attached to the tap member.

The provision of such a retaining device provides special advantages when it is desired to attach a night drainage apparatus to the tap. A night drainage apparatus is a large capacity drain or collector that attaches to the tap, to provide a larger overall collection capacity that reduces the number of times that the user has to wake up during the night to empty the collected liquid. The retaining device of the present invention can avoid accidental closing of the tap resulting from involuntary movement of the user while asleep, and thereby ensure continuous drainage into the night drainage apparatus.

(c) The tap is disposed in a weld seam of the drainage bag, such that the drain passage is through the weld seam, rather than through a wall of the bag. Preferably, the tap is disposed in the weld seam that delimits the bottom of the bag.

Disposing the tap in the weld seam can enable more complete drainage of liquid from the bag than if the tap communicates through a wall of the bag.

Preferably the housing has an external shape suitable for integration into the pouch seam. The housing may have opposed outer faces for joining to plastics film that form the front and rear walls of the drainage bag. The housing may narrow at its opposite ends to facilitate sealing between the pouch walls, especially at the opposite ends.

(d) An outlet closure is provided on the tap member, the outlet closure being distinct from, yet captive on, the tap member. The outlet closure may be made of a softer material than the tap member. The outlet closure may comprise a closure portion for blocking the outlet end of the tap member, a mounting loop that is captive around the tap member, and a strap portion joining the closure portion to the mounting loop. The outlet closure may further comprise a handle which, in use, permits manipulation of the tap member and/or the outlet closure.

Although the above aspects have been described independently, additional advantages may be achieved by using any two or more, or all, of the above aspects in combination. Although various features and aspects have been identified above and in the appended claims, protection is claimed for any novel feature or idea described herein and/or illustrated in the drawings, whether or not emphasis has been placed thereon.

### DESCRIPTION OF THE DRAWINGS

FIG. 1 is a schematic sectional view showing a tap attached to a drainage bag, the tap member in the closed position.

FIG. 2 is a schematic sectional view similar to FIG. 1, but showing the tap member in an open condition.

FIG. 3 is a schematic perspective underside view of the tap in isolation (and omitting the drip cap for brevity).

FIG. 4 is a schematic perspective underside view of the tap in isolation (and showing the drip cap in one blocking position).

FIG. 5 is a schematic side view of the tap member with the drip cap in isolation.

### DETAILED DESCRIPTION OF PREFERRED EMBODIMENT

A urine drainage bag 10 (e.g., a urostomy bag or a urine leg bag) comprises front and rear walls 12 of flexible plastics film welded together around a mutual peripheral seam 14. A tap 16 is included in the portion of the seam 14a delimiting the bottom of the collection chamber 18 within the bag 10. The tap 16 is disposed generally between the front and rear walls 12.

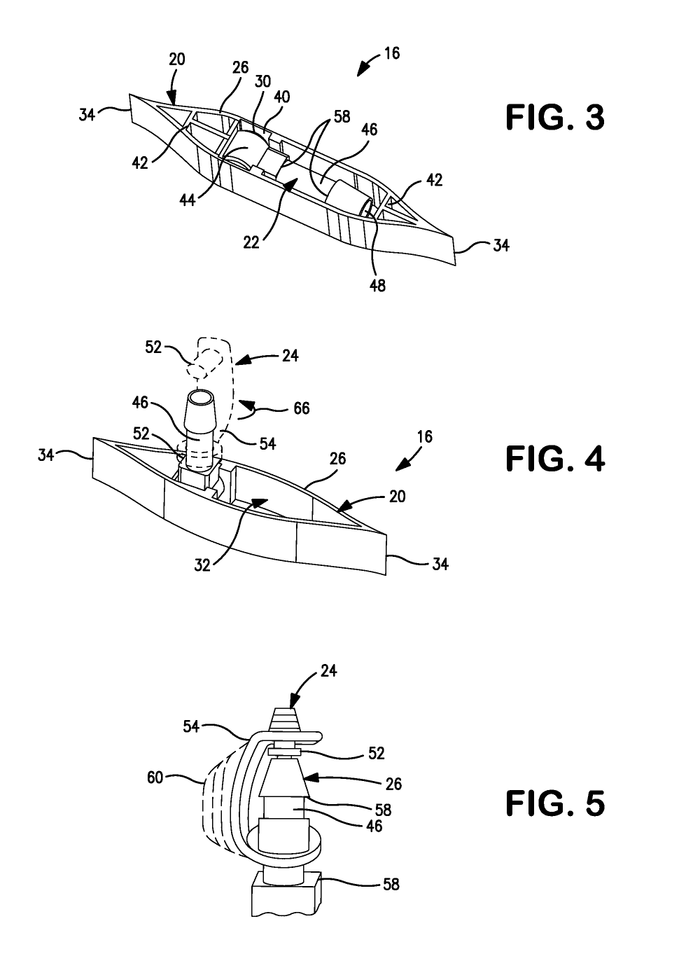

The tap 16 comprises a housing 20, a tap member 22, and an outlet closure in the form of a drip cap 24. The housing 20 comprises side walls 26 that encompass an elongate chamber 32 for receiving the tap member 22. The side walls 26 are configured to be attached in face-to-face relation to the front and rear walls 12 of the bag 10, and the side walls 26 define a boat-like shape around the chamber 32, with tapered ends 34 that narrow to a tip. The tapered shape aids sealing of the housing 20 with respect to the front and rear walls 12 of the bag 10, especially at the seam junctions at the ends 34 of the housing 20 where the portion of the seam 14a divides on either side of the housing 20. The tapered shape also provides a generally smooth profile of the housing 20 within the peripheral seam 14, so that the pouch 10 is comfortable to wear, and does not have any prominent projecting edges likely to be visible through the wearer's clothing. The particular profile of the housing 20 may be configured to suit an intended application. For example, FIG. 3 illustrates a generally straight-side profile with tapered ends. FIG. 4 illustrates a more leaf-like profile with continuously curved sides.

The housing 20 further comprises a top wall 36 with an orifice 38 towards one end. Below the orifice 38, the internal shape of the housing 20 is profiled to define a pivot socket 40. The housing 20 may further comprise one or more transverse wall portions 42 that strengthen the housing 20.

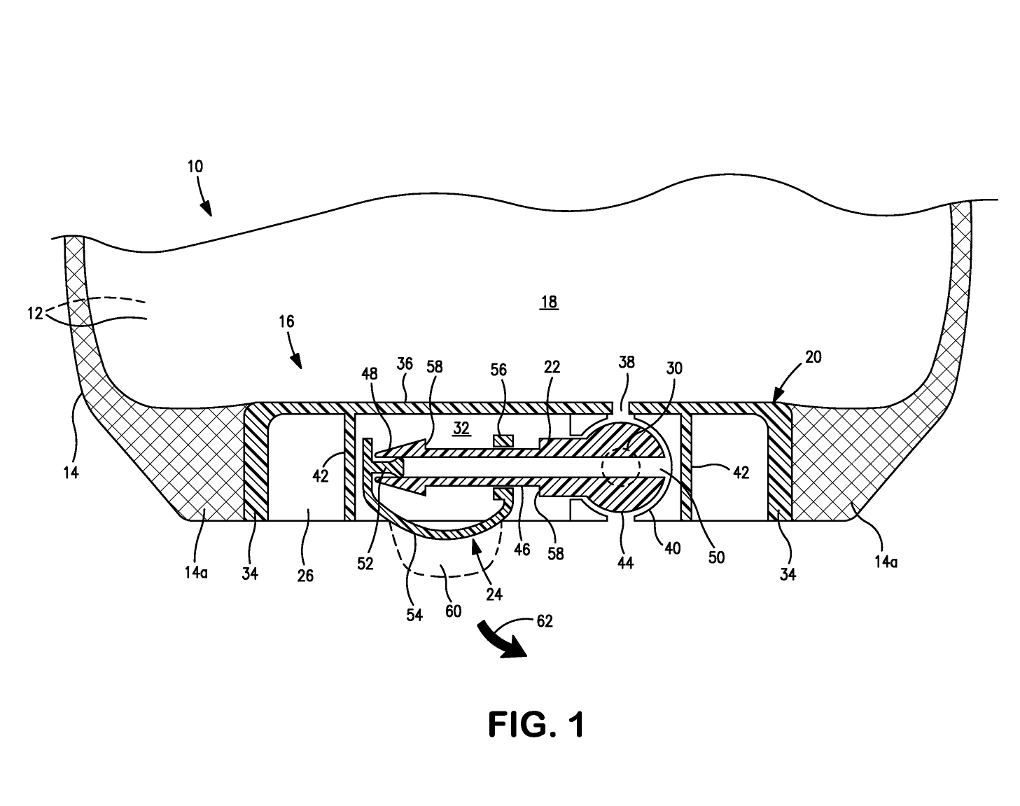

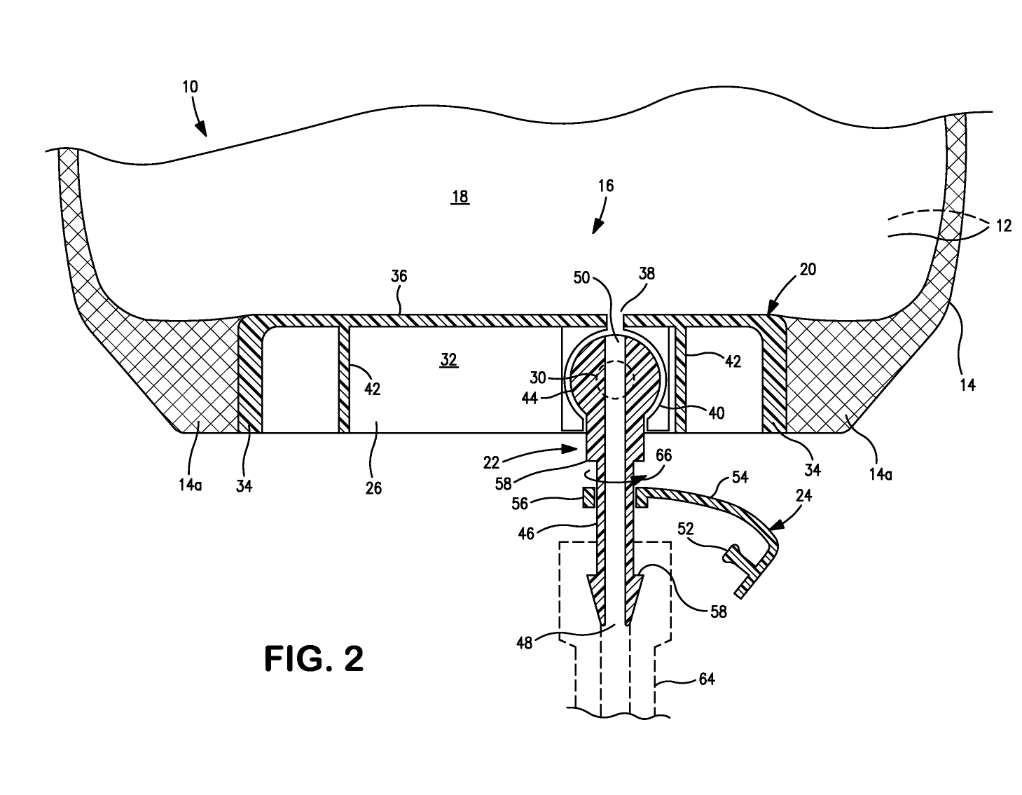

The tap member 22 generally comprises a barrel portion 44 from which extends a tubular stem 46 having an outlet orifice 48. The barrel portion 44 is configured so that it can be snap-fitted into the pivot socket 40 of the housing 20, such that the tap member 22 is pivotably movable about a pivot axis that is generally perpendicular to the elongate direction of the chamber 32. The tap member 22 is pivotably movable between a closed condition as shown in FIGS. 1 and 3, and an open condition as shown in FIGS. 2 and 4. In the open position, the tap member 22 is deployed to project substantially outside the housing 20, for example, downwards. In the closed condition, the tap member 22 is stowed at least partly within the chamber 32 such that more of the tap member 22 is received within the housing 20 than when in the open position. In the illustrated form, the tap member 22 is stowed substantially entirely within the chamber 32 when in the closed position, such that substantially no part of the tap member 22 projects beyond the confines of the housing 20. The tap 16 may be referred to as a foldaway tap, in that the tap member 22 is stowed substantially into the housing 20.

The barrel portion 44 fits tightly against the top wall 36 at orifice 38, to form a liquid tight seal that prevents leakage of liquid at the interface between the top wall 36 and the barrel portion 44. The barrel portion 44 is itself hollow, and includes a barrel orifice 50 that is generally in register with the orifice 38 of the housing 20 when the tap member 22 is moved to its open position, to allow liquid flow from the orifice 38, through the barrel orifice 50, through the stem 46 to exit via the outlet 48.

The housing 20 and the tap member 22 may be made of molded plastics. The tap body is preferably a soft plastic suitable for welding and the stem is preferably more rigid, such that the housing 20 and tap member 22 have well defined, close-fitting, shapes that form a liquid tight seal as explained above.

The drip cap 24 generally comprises a closure 52, a connecting strap 54, and mounting loop 56, integrally molded of flexible plastics material that may be softer than the plastics material of the housing 20 and/or the tap member 22. The closure 52 may be configured to plug and/or cap the outlet orifice 48 when the closure 52 is fitted to the outlet orifice 48, in order to prevent any dripping of urine from the tap member 22. The drip cap 24 is mounted captively on the tubular stem 46 of the tap member 22 by means of the mounting loop 56. The mounting loop 56 forms a generally tight fit around the tubular stem 46, such that the drip cap 24 is rotatable and/or slidable stiffly with respect to the tubular stem 46, but is normally held in place by friction and/or an interference fit, between the mounting loop 56 and the tubular stem 46. The external surface of the tubular stem 46 comprises one or more stop formations 58 that limit the degree of movement of the mounting loop 56, and keep the mounting loop 56 captive.

The drip cap 24 may be molded independently of the tap member 22, and later fitted to the tap member 22 during assembly of the tap 16. Alternatively, the drip cap 24 may be molded in position on the tap member 22, for example, using a multi-shot molding technique and non-compatible plastics that do not mutually adhere to each other during molding.

The tap 16 is intended to be assembled into a finished unit prior to integration into the drainage bag 10. This enables the tap 16, if desired, to be tested prior to manufacture of the drainage bag 10 (or a random sample of taps may be tested in order to reduce testing overhead). Assembly of the tap 16 prior to attachment to the drainage bag 10 is also generally more efficient and less expensive, since it is much easier to manipulate the tap components when in isolation. This is especially the case for the present design, which comprises three parts (namely the housing 20, the tap member 22 and the drip cap 24).

Moreover, the portions of the housing 20 that are, in use, attached to the drainage bag 10 are clear and unobstructed on both sides, which means that the tap 16 can be fixed to the drainage bag 10 by automated machinery, even in the fully assembled condition of the tap 16. The housing 20 may be attached in the portion of the seam 14a of the drainage bag 10 by any suitable attachment technique, such as welding (e.g,. heat welding, ultrasonic welding, laser welding, or impulse welding) or bonding (e.g,. adhesive bonding or solvent bonding).

As can be seen in FIG. 1, in use in the closed condition of the tap 16, i.e., with the tap member 22 rotated to be received substantially within the housing 20, and the closure 52 of the drip cap 24 fitted to the outlet orifice 48, the tap 16 has a very low profile that is accommodated substantially within the profile of the drainage bag 10. The tap 16 does not project transversely outwardly of the drainage bag 10, and there is virtually no risk of the tap 16 catching on the user's clothing and opening accidentally. The only part of the tap 16 that projects is the connecting strap 54 of the drip cap 24.

The connecting strap 54 provides a handle which the user can grip when it is desired to open the tap 16 to drain liquid from the drainage bag 10. If desired, an optional handle tab 60 may be provided on the connecting strap 54 to increase the surface area that the user can grip. By pulling on the connecting strap 54 (as indicated by arrow 62 in FIG. 1), the user can move the tap member 22 to its open, deployed position.

Once the tap member 22 is its open position, the closure 52 of the drip cap 24 is removed from the outlet orifice 48 to start the flow of urine through the tap 16. The urine may be discharged directly into a toilet bowel or into some other suitable drain or collector. As can be seen in FIGS. 1 and 2, since the tap 16 is integrated into the portion of the portion of the seam 14a that delimits the bottom of the collection chamber 18, all of the urine can be emptied from the drainage bag 10. Although in the illustrated form the portion of the seam 14a and the top wall 36 of the housing 20 are generally planar, one or both of the seam 14a and the top wall 36 may have a funnel shape if desired to funnel liquid to the tap 16.

For night drainage, the tap member 22 is placed in its open condition, and a night drainage apparatus (not shown) is coupled to the outlet orifice 48. The night drainage apparatus typically comprises a second drainage bag (not shown) of larger capacity than the drainage bag 10, and an elongate tube 64 that couples to the tap 16. A feature of the present embodiment is the provision of a lock or retainer for retaining the tap member 22 in its open condition. This can prevent the tap 16 from accidentally closing if, for example, the user moves while asleep. Were the tap 16 to close, urine would be prevented from draining and thus back-up inside the drainage bag 10, risking overflow, infection and skin irritation.

Various retainer configurations are envisaged. In one form, the drip cap 24 is used as a retainer. The drip cap 24 is movable on the stem 46 between a normal position in which the drip cap 24 does not block entry of the tap member 22 into the chamber 32, and a blocking position in which the drip cap 24 does obstruct entry of the tap member 22 into the chamber 32. For example, the drip cap 24 may be angularly movable around the stem 46. The normal (non-blocking position) may be the position shown in FIGS. 1 and 2, in which the connecting strap 54 extends generally in the plane of the drainage bag 10 (and/or in the plane defined by the chamber 32 and the tap member 22 when open). The blocking position may be that shown in FIG. 4, in which the drip cap 24 is rotated (as indicated by arrows 66 in FIGS. 2 and 4) around the axis of the stem 46 through about 90 degrees. In the blocking position, the connecting strap 54 of the drip cap 24 obstructs closing movement of the tap member 22 by abutting one of the side walls 26 if the tap member 22 is accidentally urged closed. The friction engagement between the mounting loop 56 and the stem 46 holds the angular position of the drip cap 24 with respect to the tubular stem 46, to keep the drip cap 24 in the blocking position. In order to disengage the retainer, the user rotates the drip cap 24 back to its non-blocking position, thereby allowing the tap member 22 to be closed.

Additionally or alternatively, the housing 20 can co-operate with the connector of a night drainage apparatus, in a similar abutting manner, to prevent closing of the tap member 22 while the connector of the night drainage apparatus remains coupled to the tap member 22.

It will be appreciated that taps 16 with different tap members 22 may still use the same design of housing 20. For example, different tap members 22 may have different configurations of outlet orifice 48 for coupling to different types of night drainage adapters. The housing 20 may be a universal component that can be used in different designs of tap 16, thus simplifying manufacture of different drainage bags 10 using automated apparatus. The width pf the housing 20 helps to prevent twisting of the drainage bag 10 thereby avoiding potential problems for the user.

The housing 20 and tubular stem 46 may be molded independently, however, a preferred method would be as described for the drip cap 24 wherein it is molded in a two part process using two different materials, as in mold assembly.

Although the invention is especially suited to a drainable urine drainage bag, it will be appreciated that the invention may be used with any drainage bag for collecting body liquids.

Finally, it is emphasized that the foregoing description is merely illustrative of a preferred form of the invention. Many modifications, improvements and equivalents may be used within the scope and/or principles of the invention.

I claim:

1. A tap for a drainage bag, the tap comprising a housing; and
a tap member mounted to the housing, the tap member being pivotably movable with respect to the housing between:
an open, deployed position in which at least a first portion of the tap member projects from the housing; and
a closed, stowed position in which more of the tap member is received within the housing than in the open position.

2. The tap according to claim 1, wherein the housing comprises an elongate chamber for receiving the tap member when in the closed position, and wherein the tap member is pivotably movable about a pivot axis that is generally perpendicular to the elongate direction of the chamber.

3. The tap according to claim 1, wherein substantially no part of the tap member projects from the housing when in the closed position.

4. The tap according to claim 1, wherein the housing is configured for attachment between front and rear walls of the drainage bag, the housing comprising generally oppositely outwardly facing side wall surfaces for face-to-face contact with the walls of the drainage bag.

5. The tap according to claim 4, wherein the housing has tapered ends.

6. The tap according to claim 1, further comprising a handle portion for enabling a user to pull on the tap member when the tap member is in the closed position.

7. The tap according to claim 6, wherein the handle portion forms part of a member distinct from, and captive on, the tap member.

8. The tap according to claim 1, further comprising a retaining device for retaining the tap member in the open position.

9. The tap according to claim 8, wherein the retaining device is movable between a non-activated position and an activated position.

10. The tap according to claim 8, wherein in the activated position, the retaining device blocks movement of the tap member into the closed position.

11. The tap according to claim 1, further comprising an outlet closure distinct from the tap member, the outlet closure comprising:
a closure portion for closing an outlet aperture of the tap member;
a mounting portion distinct from and captively retained on the tap member; and
a flexible strap portion coupling the closure portion to the mounting portion.

12. The tap according to claim 11, wherein the mounting portion comprises a closed loop that extends around the tap member.

13. The tap according to claim 11, wherein the strap provides a handle for pulling the tap member from its closed position.

14. The tap according to claim 11, wherein the outlet closure is movable between a blocking position in which the outlet closure blocks movement of the tap member to its closed position, and a non-blocking position in which the outlet closure does not block movement of the tap member to its closed position.

15. A tap for a drainage bag, the tap comprising:
a housing;
a tap member mounted to the housing, the tap member being pivotably movable with respect to the housing between an open position and a closed position; and
a retaining device for retaining the tap member in the open position.

16. The tap according to claim 15, wherein the retaining device comprises a member movable between a blocking position in which the retaining device blocks movement of the tap member to its closed position, and a non-blocking position in which the retaining device does not substantially block movement of the tap member to its closed position.

17. The tap according to claim 15, wherein the retaining device is mounted on the tap member.

18. The tap according to claim 15, wherein the retaining device comprises an outlet closure carried on the tap member.

19. A tap for a drainage bag, the tap comprising:
a housing;
a tap member mounted to the housing, the tap member having an outlet orifice and the tap member being pivotably movable with respect to the housing between an open position and a closed position; and
an outlet closure carried by the tap member, the outlet closure being distinct from the tap member, and the outlet closure comprising:
a closure portion for closing an outlet orifice of the tap member;
a mounting portion distinct from and captively retained on the tap member; and
a flexible strap portion coupling the closure portion to the mounting portion.

20. The tap according to claim 19, wherein the closure portion is configured to be removably fitted to close the outlet orifice.

21. A drainage bag for body liquids, the drainage bag comprising:
a front wall;
a rear wall attached to the front wall along a peripheral seam; and
a tap for controllably draining liquid from the bag, wherein the tap is disposed between the front and rear walls, the tap being integrated into the peripheral seam at a lower region of the drainage bag.

22. The drainage bag according to claim 21, wherein the tap is attached to the front and rear walls by one or more of: heat welding, ultrasonic welding, laser welding, impulse welding, adhesive bonding, and solvent bonding.

23. The drainage bag according to claim 21, wherein the tap comprises a tap member mounted to a housing, the tap member being pivotably movable with respect to the housing, between an open position and a closed position.
